(19) United States
(12) Patent Application Publication
(10) Pub. No.: US 2001/0021384 A1
(43) Pub. Date: Sep. 13, 2001

(54) Mucosally targeted immunization

(76) Inventors: Therese Jourdier, Francheville (FR); Catherine Moste, Charbonnieres les Bains (FR); Bernard Meignier, Thurins (FR)

Correspondence Address: Michael S. Greenfield, McDonnell Boehnen Hulbert & Berghoff, 32nd Floor, 300 S. Wacker Drive, Chicago, IL, 60606, US

(21) Appl. No.: 09/746,581

(22) Filed: Dec. 21, 2000

(30) Foreign Application Priority Data
Jun. 26, 1998 (FR) 98/08354
Jun. 28, 1999 (WO) PCT/FR99/01554

(51) Int. Cl. A61K 39/00; A61K 39/38; A61K 39/09; A61K 39/10

(52) U.S. Cl. 424/184.1; 424/244.1; 424/253.1

(57) ABSTRACT

The invention concerns the use of an immunogen specific of a pathogenic agent with a gateway in the buccal mucous membrane region, for producing a vaccine composition to be administered in the floor of the mouth in a human being so as to develop directly a local response in IgA antibodies and in B cells secreting IgA in the buccal mucous membrane, saliva and ganglions draining said mucous membrane. The invention also concerns a vaccine composition capable of being applied in the floor of the mouth in a human being to induce local and systemic immunity in IgA antibodies, substantially consisting of a material adhering or not to the buccal mucous membrane and containing an immunogen specific of the pathogenic agent with a gateway into the buccal mucous membrane.

The present invention relates to an immunization method which makes it possible to induce a local immune response in the buccal mucous membrane region. It also relates to the use of immunogens for preparing vaccine compositions intended to be administered according to said method.

### STATE OF THE ART

The main route of transmission of the AIDS virus consists of the mucous membranes, in particular genital and rectal mucous membranes, or even buccal mucous membranes. From these mucous membranes, the virus rapidly disseminates toward the draining lymph nodes, before entering the peripheral blood.

As in the case of other pathogenic agents (virus, bacterium, etc.) with a mucosal gateway, the induction of immunity capable of blocking the virus as it enters into the mucous membrane or in the first steps of its dissemination in the lymph nodes appears to be important.

Most studies are generally aimed toward obtaining systemic immunity which can be detected by the serum antibody titer, as described by Jian-Ming X. et al. (vaccine, 993-1000, 1996) Kim J. J. et al. (vaccine, 879-883, 1997), Anderson et al. (The Journal of Infectious Diseases, 960-969, 1989), David D. et al. (vaccine, 1661-1669, 1997), Raskisov et al. (U.S. Pat. No. 4,368,191) and Transgene (FR2751879).

The targeting of local immunity of the mucosal type can be demonstrated by the presence of specific antibodies contained in the mucous membranes or the secretions (Thibodeau L. et al., Aids Research and Human Retrovirus, 1379, 1992; Russel M. W. et al., Reviews of infectious diseases, 5440-5446, 1988).

The studies of Lehner et al., (Nature Medicine, 767-775, 1996) carried out on rhesus monkeys have shown that it is possible to induce local immunity with respect to the SIV virus by performing a deep subcutaneous injection in the pelvic region close to the iliac lymph nodes. This immunization causes the induction of IgA- and IgG-type antibodies in rectal and urinary fluids and in the serum. Such an immunization method cannot, however, be used in humans.

Letchworth G. J. et al. (U.S. Pat. No. 5,462,734) teaches that an intramuscular injection (the injection site is not specified) of a glycoprotein induces a systemic response only, and the booster given in mucous membranes enables local mucosal immunity to then be obtained. The teas do not target the oral cavity. The production of secretary IgA antibodies as well was not reported.

Gaffar A. et al. (U.S. Pat. No. 3,931,398) put forward the hypothesis that an injection of an anti-carie vaccine in the oral cavity would make it possible to induce local immunity. There is, however, no report of a test directed toward measuring the response in respect of IgA antibodies.

L. Thibodeau et al. (C.R. Acad. Sci. Paris, 389-394, 1991) report that the repeated topical application of HIV gp160 to the oral mucous membranes of rabbits, followed by a parenteral injection, makes it possible to obtain systemic and local immunity of the IgA type. The topical application to the oral mucous membranes does not, however, make it possible to obtain a systemic response of the IgA type, since only the booster via the parenteral route provides this effect. In actual fact, there are no grounds for saying that the results can be applied to humans.

J. Hinkula et al. (Vaccine, 874-878, 1997) show that the injection of HIV DNA under the control of a CMV promoter into the tongue or into the gums of mice makes it possible to obtain a systemic IgA response detected in peripheral blood. The production of an IgA response in secretions is not reported. In actual fact, the injection routes cannot really be applied to humans.

The aim of the present invention is, therefore, to provide a simple and effective route of administration which makes it possible to induce directly local and systemic immunity, in particular to induce locally immunity in respect of IgA antibodies and B cells which secrete IgAs, in the mucous membrane and in the buccal secretions in humans, and in the lymph nodes which drain them.

### SUMMARY OF THE INVENTION

The invention is therefore directed toward the use of an immunogen specific for a pathogenic agent with a gateway into the buccal mucous membrane region, for producing a vaccine composition intended to be administered in the floor of the mouth in humans so as to develop directly a local response in respect of IgA antibodies and of B cells which secrete IgAs in the buccal mucous membrane, in the saliva and in the lymph nodes draining said mucous membrane.

One particular aim of the invention is to provide such a route of immunization in order to develop local immunity against the AIDS virus HIV).

Another aim of the invention is to induce simultaneously systemic and local immunity in respect of antibodies via at least a single route of administration. The term “local immunity” is intended to mean an IgA, IgG and/or IgM response in the buccal mucous membrane, in the saliva and in the lymph nodes draining said mucous membrane, whereas systemic immunity relates to an IgA, IgG and/or IgM response detected in peripheral blood.

Yet another aim or the invention is to provide a mode of immunization which may be very advantageously combined with a conventional immunization in order to supplement it and, generally, with any other type of immunization, local or systemic.

Another aim is to provide a route which allows the injection of significant volumes of a vaccine.

Another subject of the present invention consists of a vaccine composition which can be applied in the floor of the mouth in humans in order to induce local and systemic immunity in respect of IgA antibodies, and which consists substantially of a material which adheres to the buccal mucous membrane and which contains at least one immunogen specific for a pathogenic agent with a gateway into the buccal mucous membrane region.

Finally, a last subject of the present invention relates to a vaccine composition which can be deposited in the floor of the mouth in humans in order to induce local and systemic immunity in respect of IgA antibodies, and which consists of a material which does not adhere to the buccal mucous membrane, which is degraded on contact with the secretions and which contains at least one immunogen specific for a pathogenic agent with a gateway into the buccal mucous membrane region, characterized in that this material contain invasive elements which promote the penetration of the immunogen through the buccal mucous membrane.

### DETAILED DESCRIPTION OF THE INVENTION

The subject of the present invention is thus in particular the development of a local and systemic response in the buccal mucous membrane and in the lymph nodes which drain it, by administering an immunogenic composition in the floor of the mouth in humans. Against all expectations, this route makes it possible to obtain directly a local and systemic response, something which had not been demonstrated before.

The invention applies both to the field of prophylaxis (vaccines) and to the field of active immunotherapy. The term “immunogenic composition” covers, therefore, compositions for prophylactic purposes, in particular vaccines, and compositions for curative purposes in which the immunogen is of antigen type.

It is thus possible to administer the immunogenic composition by parenteral injection, e.g. by sublingual injection in the floor of the mouth. The production of IgA, IgG and/or IgM immunoglobulins is induced locally in this mucous membrane and in the secretions (saliva), and regionally in the lymph nodes which drain this region and which induce the production of B cells which secrete said antibodies, while at the same time inducing systemic immunity. This immunity is capable of inducing protection against the entry and dissemination of the pathogen under consideration from this buccal mucous membrane region.

It is also possible to administer the immunogenic composition by depositing it in the floor of the mouth, even though this technique is not reputed to give the best results. Preferably therefore, a bioadhesive or capsule which releases the immunogenic composition is placed in the floor of the mouth. Bioadhesives specific for the oral mucous membrane are well known to those skilled in the art, and have been in particular described by the company 3M Pharmaceuticals (Drug Dev. & Indus Phar., 2809-2821, 1994), Taylan B. et al. (J. Controlled Release, 11-20, 1996), Nakane S. et al. (Pharmaceutical Dev. & Tec, 251-259, 1996), Efentakis M. et al. (S.T.P. Pharma sciences, 227-232, 1998), and Diarra M. et al. (Biomaterials, 1523-1527, 1998). Capsules capable of releasing a pharmacological composition according to desired kinetics are also well known to those skilled in the art. By way of nonlimiting indications, mention may thus be made of Save T. et al., J. of Pharmacy & Pharmacology, 192-195, 1994; WO9618389; WO9908662; WO9922767; WO9921586; WO9917744; EP913149; EP874642 and WO9908729.

Preferably, the bioadhesives or capsules used contain or are coupled to a system which promotes the penetration of the immunogenic composition through the floor of the mouth, for example by means of abrasive particles, possibly at the surface of the bioadhesives or capsules, or by means of a particular formulation of the immunogenic composition, such as lyposomes for example. By way of indication, mention may be made of the article by Gandhi et al. (Advanced Drug Delivery Reviews, 43-74, 1994).

The invention also relates, therefore, to any vaccine composition which can be applied in the floor of the mouth in humans in order to induce local and systemic immunity in respect of IgA antibodies, and which consist substantially of a material which adheres to the buccal mucous membrane, such as one of those described in the above literature, and which contains at least one immunogen specific for a pathogenic agent with a gateway into the buccal mucous membrane region. This adhesive material can contain or be coupled to a system which promotes the penetration of the immunogen through the buccal mucous membrane, such as abrasive particles consisting of, for example, hydroxyapatite or other salt crystals, or a particular formulation of the immunogen.

The invention also relates to a vaccine composition which can be deposited in the floor of the mouth in humans in order to induce local and systemic immunity in respect of IgA antibodies, and which consists of a material which does not adhere to the buccal mucous membrane, which is degraded upon contact with the secretions and which contains at least one immunogen specific for a pathogenic agent with a gateway into the buccal mucous membrane region, characterized in that this material contains invasive elements which promote the penetration of the immunogen through the buccal mucous membrane. This composition can therefore take the form of a capsule made with starch, with maltodextrins or other polysaccharides containing invasive elements, such as crystals of a salt or of hydroxyapatite, for example

Against all expectations, the route of immunization according to the invention makes it possible in particular to obtain secretary IgAs in the form of dimers of antibodies linked to the secretary component, having the property of being particularly resistant and, as a result, likely to be more suitable for neutralization activity The definitions of secretary IgAs adopted by Thibodeau L. (above) and Russel M. W. (above) are incorporated into the description of the present invention, by way of reference.

A first subject of the invention is, therefore, the use of an immunogen specific for a pathogenic agent with a gateway into the buccal mucous membrane region, for producing an immunogenic composition intended to be administered so as to develop a local response in respect of IgA, IgG and/or IgM antibodies and of B cells which secrete IgAs, IgGs and/or IgMs in the buccal mucous membrane, in the saliva and in the lymph nodes draining this mucous membrane, in particular in the submaxillary lymph nodes. Sublingual injection in the floor of the mouth is the preferred means in particular for enabling the best recruitment of B cells in the lymph nodes draining the buccal mucous membrane region. In addition, the amounts of immunogenic composition injected can range from 0.1 to 3 ml, in particular 0.5 to 2 ml, for example.

Among the pathogenic agents to which the invention can be applied, mention may be made most particularly of: the HIV virus, herpesviruses, in particular herpes simplex virus, candidae, hepatitis viruses, in particular hepatitis A virus, picornaviridae, in particular enteroviruses such as poliomyelitis virus, reoviruses, in particular rotaviruses, adenoviruses, human papillomavirus, periodontosis, cytomegalovirus, Epstein-Barr virus, and all pathogens transmitted via aerosols, such as M. tuberculosis, N. meningitidis, Streptococcus type B, S. pneumoniae or B. pertussis, for example.

It should he noted that this administration method is not limited to inducing a local response, and may also make it possible to induce, at the same time, a systemic response, the two actions combining and complementing, or even reinforcing, each other particularly advantageously.

Consequently, the use in accordance with the invention is also directed toward developing, in addition to a local response in respect of IgA, IgG and/or IgM antibodies and of B cells which secrete said antibodies, a systemic response of IgA, IgG and/or IgM type (antibodies and secreting B cells). Preferably, the local and systemic response is of the IgA and IgG type at least.

Without it being necessary to state it each time, it goes without saying that when reference is made to a response in respect of IgGs, IgMs or IgAs, antibodies and secreting B cells, it is a response specific to the immunogen used.

Another subject of the invention is the method for immunizing against pathogenic agents as described above, consisting in administering, by any means known per se, the appropriate immunogenic composition so as to induce a local response as described above. Without it being necessary to restate it each time, the immunization method can have each of the characteristics, alone or in combination, set out herein in the context of the use. It will be recalled that sublingual injection in the floor of the mouth is the preferred means.

In the context of the immunization method and use, it may also be specified that the invention applies to all types of immunogenic composition, and in particular vaccines, that are known, whether they are of the conventional type or of the recombinant type. As is known per se, the compositions, e.g. vaccines, of conventional type group together compositions, e.g. vaccines, which are whole, live, attenuated or inactivated, and subunits (proteins or peptides), these conventional compositions, e.g. conventional vaccines, possibly being adjuvanted and possibly being in combined form grouping together various valences and/or various immunogenic forms of the same valence. The recombinant compositions, e.g. recombinant vaccines, group together live vectors which express one or sore immunogens of the pathogen under consideration, and polynucleotide plasmid vectors which consist of a DNA which can be, for example, naked or included in a lyposome (see e.g. WO-A-90 11092, WO-A-93 19813, WO-A-94 21797, WO-A-95 20660), and which express one or more immunogens. With regard to recombinant live vectors, vectors which may be mentioned in particular include poxviruses, for instance the vaccinia virus and especially avian poxviruses (canarypox, fowlpox, pigeonpox, etc) such as those described in Tartaglia et al., Virol. 1992, 188:217, and adenoviruses. Since it involves a novel route of administration, it is quite obvious that the invention cannot be limited to one particular type of composition, e.g. of vaccine, but is intended to apply to all types of immunogenic composition, e.g. of vaccine, and to all available compositions, e.g. vaccines, which can be used via this route.

Similarly, the immunization protocol will depend on the type of composition or of vaccine, or on the composition or vaccine, used. It would include the number of administrations conventionally used for a given vaccine, which, generally, will correspond to more than one administration, in particular from 2 to 4. A person skilled in the art is, in any case, perfectly capable of determining, through routine tests, the optimum number of administrations (e.g. primary vaccination and booster).

This targeted immunization may also be combined with conventional systemic immunization with the same composition or another composition against the same pathogen.

It is also possible to combine with it, in the same host, an immunization protocol targeting the recto-genito-urinary mucous membrane, comprising the administration of a composition immunogenic against the same pathogen, which may be identical or different, in particular of the same composition, preferably by parenteral injection into the thigh (one or both right and left lower limbs), preferably via the intramuscular route, in particular into the quadriceps, especially into the rectus femoris muscle. This immunization of the recto-genito-urinary mucous membrane is directed toward inducing a local response in respect of IgA, IgG and/or IgM antibodies, and of B cells which secrete said antibodies in the mucous membrane and the lymph nodes draining this mucous membrane, in particular in the external and internal iliac lymph nodes and in the inguinal lymph nodes (it may also be accompanied by a systemic reaction).

Such a combination is particularly useful for preventing or treating an infection by a pathogen with both the buccal and recto-genito-urinary gateways. Mention may be made in particular of the HIV virus and herpesviruses.

Consequently, according to an advantageous development of the present invention, the use of an immunogen specific for a given pathogen is directed toward the production of, firstly, an immunogenic composition intended to be administered in humans in the floor of the mouth so as to induce a response in respect of IgA, IgG and/or IgM antibodies and of B cells which secrete said antibodies in the buccal mucous membrane, in the saliva and in the lymph nodes draining this mucous membrane, in particular the submaxillary lymph nodes, and preferably by sublingual injection in the floor of the mouth and, secondly, an immunogenic composition, which may be identical to or different from the previous one, intended to be administered in the same host via the parenteral route into the thigh, preferably via the intramuscular route, especially into the quadriceps, in particular into the rectus femoris muscle, so as to induce a local response in respect of IgA, IgG and/or IgM antibodies and of B cells which secrete said antibodies in the recto-genito-urinary mucous membranes.

The corresponding immunization method provides, therefore, for this double administration. This immunization which targets the recto-genito-urinary mucous membranes is also accompanied by systemic immunization which combines advantageously with that resulting from the immunization which targets the buccal mucous membrane. These two local immunizations can also be combined with conventional systemic immunization, using the same composition or different compositions directed against the same pathogen. The immunization method and use in accordance with the invention have a preferred application in the context of vaccination against the HIV virus. A particular example is the use of a vaccine grouping together a vector expressing HIV gp120/gp160 and of [sic] the gp120/gp160 glycoprotein subunit of this same virus. A particular example is described below.

In the context of the present invention, it is thus envisaged to use this anti-HIV vaccine for administration directed toward the buccal mucous membrane as described above, optionally combined with intramuscular administration in the thigh and/or conventional systemic immunization, e.g. by intramuscular injection into the deltoid.

Finally, a subject of the invention is also a vaccine composition comprising a vaccine against a pathogen with a gateway into the buccal mucous membrane region and a pharmaceutically acceptable vehicle or excipient, this vehicle or excipient, or this composition, leading to, in conjunction with the vaccine, a local response in respect of IgA, IgG and/or IgM antibodies and of B cells which secrete said antibodies in this mucous membrane region, when the composition is in particular administered in the floor of the mouth. In the context of this vaccine composition, the characteristics get out herein with regard to the other subjects of the invention can be taken singly or in combination.

The invention will now be described in greater detail with the aid of embodiments taken by way of nonlimiting examples. It should be clearly understood that the invention defined by the appended claims is not limited to the particular embodiments given in the description above, but encompasses the variants thereof which depart neither from the context nor from the spirit of the present invention.

### EXAMPLE I-VACCINE

I-1. vCP205 recombinant live vaccine, ALVAC-HIV:

vCP205 is an ALVAC canarypox virus, the construction of which is described in Example 14 of WO-A-95 27507, to which a person skilled in the art may refer. It is capable of expressing the env, gag and pro genes of the HIV-1 virus. These genes are inserted into the C3 locus and are regulated by the H6 and I3L promoters of the vaccinia virus

A plasmid pHIV32 containing the expression cassettes for the gene of the env glycoprotein gp120 MN (plus the transmembrane portion of gp41 LAI) and the LAI strain genes encoding gag and the protease pro was used as a donor plasmid in an in vivo recombination procedure in order to produce vCP205. These cassettes were inserted into the C3 locus, between the ALVAC flanking sequences, in a 5′-5′ configuration, and linked to the H6 and I3L promoters.

The vCP205 was produced on chicken embryo fibroblasts in DMEM-Ham F12 medium without serum, supplemented with lactoglutamate and clarified by centrifugation. The mean titer was 10^{8.0} CCID₅₀/ml on QT35 cells. The vaccinating solutions are prepared by dilution in PBS (“phosphate buffered saline”) with Ca⁺⁺ and Mg⁺⁺.

I-2. Gp160MN/LAI-2 subunit vaccine

The subunit produced is a hybrid gp160 subunit obtained from a pox vector.

A vaccine vector VVTG9150 is used to produce the gp160. This vector encodes a hybrid soluble gp160 in which the gp120 portion is derived from the MN-HIV-1 strain and the gp41 portion originates from the LAI isolate. The corresponding DNA sequences were fused with the aid of an artificial SmaI restriction site, modifying neither of, the two amino acid sequences of gp120 and gp41. The construction is briefly described below.

The sequence encoding gp120 MN was amplified from SupT1 cells infected with HIV-MN, by the PCR technique, using oligonucleotides which introduce an SphI restriction site and an SmaI site respectively immediately downstream of the sequence encoding the leader peptide and upstream of the cleavage sites located between gp120 and gp41.

The sequence encoding gp41 was thus produced: the complete coding sequence of LAI HIV-1 env was placed under the control of the pH5R promoter of the vaccinia virus. Several modifications were made. An SphI restriction site was created immediately downstream of the sequence encoding the leader peptide, without modifying the amino acid sequence. An SmaI restriction site was also created immediately upstream of the sequence encoding the cleavage sites between gp120 and gp41, without modifying the amino acid sequence. The two cleavage sites at position 507-516 (amino acid numbering according to Myers et al. in: Human retroviruses and AIDS (1994) Los Alamos National Lab (USA)) were mutated (original sequence: KRR . . . REKR mutated to QNH . . . QEHN). The sequence encoding the transmembrane hydrophobic peptide IFIMIVGGLVGLRIVFAVLSIV (amino acids 689-710 according to Myers et al. above) was deleted. A stop codon was introduced in place of the second E codon of the sequence encoding PEGIEE (amino acids 735-740 according to Myers et al.), i.e. the 29^{th} amino acid of the intracytoplasmic domain.

The plasmid into which the LAI sequence was inserted between the homologous regions of the TK gene of the vaccinia virus was cleaved with SphI and SmaI, and then linked to the gp120 MN sequence. VVTG9150 was then constructed by conventional homologous recombination and propagated in order to express the gp160, according to the method conventionally used for VCP205 on BHK21 cells. The gp160 was then purified by immunoaffinity chromatography.

### EXAMPLE II-TEST 1

Two female rhesus monkeys (P9224 and P9225), already immunized via the intramuscular route (in the left or right thighs, alternatively) twice with 10^{6.5} CCID₅₀ of clarified ALVAC-HIV (vCP205), and then three times with 100 μg of gp160 MN/LAI-2 adjuvanted with OspA (“outer surface protein A” from Borrelia burgdorferi) and aluminium hydroxide, were inoculated twice, with a gap of one month, in the floor of the mouth (sublingual), with a mixture containing 10⁶ CCID₅₀ of clarified vCP205 and 100 μg of gp160 MN/LAI-2. The saliva, the urine, the vaginal and rectal secretions and the serum were analyzed by ELISA in order to detect the presence of anti-gp160 and anti-CPpp (against the canarypox virus itself) IgAs and IgGs.

One of the two monkeys (P9225) received an additional injection of the same mixture (vCP205+gp160 MN/LAI-2) in the floor of the mouth and the upper right thigh, three months after the last injection. The lymphocytes from peripheral blood and from certain lymph nodes (submaxillary, axillary, inguinal and iliac) were analyzed by ELISPOT to detect B cells which produce IgA and IgG antibodies specific for gp160 and CPpp.

The appearance of anti-gp160 and anti-CPpp IgAs of local origin could be shown in the mouthwash of the P9224 macaque after the first and second sublingual injections, and the appearance of anti-gp160 IgAs of plasmatic origin could be shown in the vaginal wash of this same monkey. The anti-gp160 specific IgG responses appeared from the first injection in most of the secretions assayed, and were maintained throughout the study.

Moreover, the sera of the two macaques showed a significant increase in the IgAs and IgGs specific for gp160 and CPpp.

Finally, preferential induction of B cells which secrete anti-gp160 and anti-CPpp IgA⁻ and IgG⁻ antibodies in the lymph nodes targeted by the immunizations, namely the submaxillary lymph nodes and the right inguinal and iliac lymph nodes, was demonstrated by ELISPOT in monkey P9225. The cells were also present in peripheral blood, but at lower frequency.

In conclusion, this test has shown the possibility of inducing a local and systemic anti-HIV-1 antibody response in rhesus monkeys after immunization close to the lymph nodes which drain the buccal and recto-genito-urinary mucous membranes.

### EXAMPLE III-TEST 2

The vaccine is a mixture containing 10^{6.3} CCID50 of vCP205 and 100 μg of gp160 subunit.

An ALVAC vector comprising no HIV sequence was also used, as a control.

Group 1: 4 monkeys (rhesus monkeys) received an injection of the vaccine mixture via the sublingual route in the floor of the mouth, 4 times, at 1 month intervals; equal volume mixture each group of macaques. The calculated mean and standard deviation were rounded to the closest unit.

The right and left lymph nodes of each category (submaxillary, axillary, inguinal, internal iliac and external iliac) were removed after sacrificing the animal, ground (the right and left submaxillary lymph nodes were pooled), and then subjected to analysis of the antibody-producing cells by the ELISPOT technique adapted from Eriksson K. et al., Journal of Immunological methods, 153: 107-113, 1992).

The results of counting B lymphocytes which secrete IgGs are given in the table which follows. Similar results were obtained with IgAs.

A systemic response mediated by IgA and IgG antibodies was also noted. The results of the response in respect of specific serum antibodies obtained in the group of monkeys immunized via the sublingual route (group 1) are listed in Table No. 2.

TABLE

| | Immuni- | Sample | | | |
|---|---|---|---|---|---|
| group | zation: | (sacrifice | | | |
| (no. of | Immuno- | at W14, | IgG+ B lymphocytes per 10⁶ | | |
| monkeys/ | genic | after 4 | mononucleated cells | | |
| group) | route | injections) | Total | gp160 | CPpp |
| 1 (n = 4) | sublingual | Blood | 167 ± 48 | 1 ± 0 | 1 ± 1 |
| | injection | | | | |
| | ALVAC- | | | | |
| | HIV (vC- | | | | |
| | P205) + | | | | |
| | gp160 | | | | |
| | MN/LAI-2 | | | | |
| | | Submax- | 1168 ± 271 | 190 ± 127 | 141 ± 63 |
| | | illary | | | |
| | | lymph | | | |
| | | nodes | | | |
| | | Axillary | 430 ± 311 | 0 ± 0 | 2 ± 2 |
| | | lymph | | | |
| | | nodes | | | |
| | | Internal | 662 ± 345 | 2 ± 1 | 2 ± 1 |
| | | iliac | | | |
| | | lymph | | | |
| | | nodes | | | |
| | | External | 996 ± 508 | 1 ± 1 | 3 ± 3 |
| | | iliac | | | |
| | | lymph | | | |
| | | nodes | | | |
| | | Inguinal | 320 ± 83 | 1 ± 1 | 2 ± 1 |
| | | lymph | | | |
| | | nodes | | | |
| 2 (n = 4) | IM | Blood | 152 ± 40 | 1 ± 0 | 2 ± 0 |
| | Injection | | | | |
| | (thigh) | | | | |
| | ALVAC- | | | | |
| | HIV (vC- | | | | |
| | P205) + | | | | |
| | gp160 | | | | |
| | MN/LAI-2 | | | | |
| | | Submax- | 575 ± 156 | 2 ± 1 | 2 ± 1 |
| | | illary | | | |
| | | lymph | | | |
| | | nodes | | | |
| | | Axillary | 1251 ± 393 | 2 ± 0 | 2 ± 1 |
| | | lymph | | | |
| | | nodes | | | |
| | | Internal | 657 ± 188 | 9 ± 4 | 4 ± 6 |
| | | iliac | | | |
| | | lymph | | | |
| | | nodes | | | |
| | | External | 752 ± 179 | 277 ± 146 | 226 ± 175 |
| | | iliac | | | |
| | | lymph | | | |
| | | nodes | | | |
| | | Inguinal | 817 ± 199 | 62 ± 88 | 11 ± 13 |
| | | lymph | | | |
| | | nodes | | | |
| 3 (n = 3) | IM | Blood | 173 ± 46 | 0 ± 0 | 3 ± 1 |
| | Injection | | | | |
| | (thigh) | | | | |
| | ALVAC | | | | |
| | vector | | | | |
| | (CPpp) | | | | |
| | | Submax- | 540 ± 148 | 0 ± 0 | 1 ± 1 |
| | | illary | | | |
| | | lymph | | | |
| | | nodes | | | |
| | | Axillary | 624 ± 132 | 0 ± 0 | 1 ± 0 |
| | | lymph | | | |
| | | nodes | | | |
| | | Internal | 612 ± 67 | 0 ± 0 | 4 ± 4 |
| | | iliac | | | |
| | | lymph | | | |
| | | nodes | | | |
| | | External | 700 ± 162 | 0 ± 0 | 248 ± 202 |
| | | iliac | | | |
| | | lymph | | | |
| | | nodes | | | |
| | | Inguinal | 531 ± 83 | 0 ± 0 | 210 ± 80 |
| | | lymph | | | |
| | | nodes | | | |

TABLE 2
Anti-gp160 antibody responses of the sera of the monkeys in group 1 before and after
immunization via the sublingual route with the ALVAC-HIV (vCP205) + gp160 mixture

| Immunization | Groups | Macaques | W0* | W4* | W6 | W8* | W10 | W12* | W14 |
|---|---|---|---|---|---|---|---|---|---|
| ALVAC-HIV (vCP205) | | 1 | 0.000 | 2.381 | 4.702 | 4.468 | 5.053 | 4.703 | 4.821 |
| 10^{6 3} CCID50 | | 2 | 0.000 | 2.671 | 4.979 | 4.777 | 5.139 | 4.882 | 5.108 |
| | | 3 | 0.000 | 2.896 | 4.821 | 4.361 | 4.973 | 4.533 | 4.828 |
| + | 1 | | | | | | | | |
| 100 μg gp160 MN/LAI | | 4 | 0.000 | 3.724 | 5.113 | 4.867 | 5.027 | 4.827 | 4.933 |
| sublingual injection | | m | 0.000 | 2.918 | 4.903 | 4.618 | 5.048 | 4.736 | 4.923 |
| | | sd | 0.000 | 0.577 | 0.180 | 0.242 | 0.069 | 0.155 | 0.134 |

*date of administering the vaccine mixture
m: mean of the serum titers
sd: standard deviation

### EXAMPLE IV-TEST 3

The anti-gp160 IgA and IgG antibodies in the serum and the mucous secretions (urine, and mouth, vaginal and rectal washes) of the four macaques of Example III injected via the sublingual route were assayed by the ELISA technique. The samples were taken before immunization (WO), and then after immunization just before the animal was sacrificed (W14). The results are expressed, for each secretion, in the form of the ratio of the specific anti-gp160 IgA or IgG activity measured at the time of sacrifice with respect to that evaluated before immunization. It was considered that a secretion was significantly positive for specific IgAs or IgGs if this ratio was greater than 3.

Comment: the specific activity of a secretion corresponds to the titer of specific IgAs or IgGs (here specific for gp160) divided by the titer of total IgAs or IgGs.

Moreover, the local or plasmatic origin of the specific IgAs or IgGs in a secretion was evaluated according to two different techniques described below.

1. Measurement of the coefficient of relative excretion of the Igs (A or G) with respect to albumin (protein of essentially plasmatic origin), according to the technique described by Belec L. et al., AIDS Research And Human Retroviruses, 12: 157-167, 1996.

2. Measurement of the coefficient of local production of the IgS (A or G), comparing the specific activity measured in she secretion to that in the serum taken at the same time, according to the technique described by Van Cott T. et al., Journal of Immunology, 160: 2000-2012, 1998.

The results are given in the tables below and show the preferential production, in the buccal secretions, of IgAs and of IgGs, the origin of which is local (secretory IgAs and IgGs).

Results

| Ratio of the specific anti- | | | Ratio of the specific anti- | | |
|---|---|---|---|---|---|
| gp160 IgG activities at W14 | | | gp160 IgA activities at W14 | | |
| versus W0 | | | versus W0 | | |
| Secretions | Monkey # | Ratio | Secretions | Monkey # | Ratio |
| Mouthwashes | 1 | 16.6 | Mouth- | 1 | 24.2 |
| | 2 | 22.3 | washes | 2 | 62.7 |
| | 3 | 13.0 | | 3 | 15.7 |
| | 4 | 21.6 | | 4 | 40.0 |
| Urine | 1 | <3 | Urine | 1 | <3 |
| | 2 | 4.5 | | 2 | <3 |
| | 3 | <3 | | 3 | <3 |
| | 4 | 5.0 | | 4 | <3 |
| Rectal | 1 | <3 | Rectal | 1 | <3 |
| washes | 2 | 3.3 | washes | 2 | <3 |
| | 3 | <3 | | 3 | <3 |
| | 4 | <3 | | 4 | <3 |
| Vaginal | 1 | 57.7 | Vaginal | 1 | <3 |
| washes | 3 | 94.4 | washes | 3 | <3 |

### EXAMPLE V-BIOADHESIVE

An amount of bioadhesive material containing 10⁸ CCID50 of vCP205 and 40 μg of gp160 subunit (see the examples above) is prepared according to the method of Kriwet B. et al. (J Controlled Release, 1-3, 1998). For this purpose, microparticles made of polyacrylate, approximately 1 mm in diameter, are synthesized. The system is stabilized with an emulsifier SpanTM 80 and TweenTM 80, dispersed in a fatty layer. The initial polymerization medium contains, therefore, droplets of emulsions and inverted micelles. The polymerization is initiated by radicals, and enables the encapsulation of vCP205 and of gp160 formulated beforehand also in liposomes. The final bioadhesive is placed in the floor of the mouth of a human being in order to induce local immunity in respect of anti-gp160 IgA, IgG and/or IgM antibodies, and of B cells which secrete said antibodies in the buccal mucous membranes, in the buccal secretions and in the lymph nodes which drain them, and systemic immunity for said antibodies and B cells.

### EXAMPLE VI-BIOADHESIVE

An amount of bioadhesive material containing 10⁸ CCID50 of vCP205 and 400 μg of gp160 subunit (see the examples above) is prepared according to the method of Li C. (Drug Dev Ind. Pharm, 919-926, 1998). For this purpose, the immunogenic composition consisting only of gp160 and of vCP205 is encapsulated in a polymer consisting of silicone and of Carbopol 974P. The adhesive particles are also coated with hydroxyapatite crystals in order to facilitate the penetration of the composition through the mucous membrane. The final bioadhesive is placed in the floor or the mouth of a human being in order to induce local immunity in respect of anti-gp160. IgA, IgG and/or IgM antibodies, and of B cells which secrete said antibodies in the buccal mucous membranes, in the buccal secretions and in the lymph nodes which drain them, and systemic immunity for said antibodies and B cells.

### EXAMPLE VII-CAPSULES

Capsules made of starches and of hydroxyapatite particles containing lyophilized antigens of the cytomegalovirus, of [lacuna] hepatitis A, or of pathogens transmitted conventionally by aerosols, such as M. tuberculosis, N. meningitidis, Streptococcus type B. S. pneumoniae, B. pertussis, for example, are prepared. These capsules are designed to dissolve slowly in the mouth. The hydroxyapatite crystals facilitate the penetration of the composition through the mucous membrane. A capsule is placed in the floor of the mouth of a human being in order to induce local immunity in respect of anti-gp160 IgA, IgG and/or IgM antibodies, and of B cells which secrete said antibodies in the buccal mucous membranes, in the buccal secretions and in the lymph nodes which drain them, and systemic immunity for said antibodies and B cells.

We claim:

1. A method of producing a local immunogenic response in a buccal mucous membrane, saliva, and lymph nodes draining said mucous membrane, the method comprising administering in the floor of a mammal's mouth a vaccine composition comprising an immunogen specific for a pathogenic agent having a gateway into the buccal mucous membrane, wherein a local IgA antibody and IgA antibody-producing B cell response is induced.

2. The method of claim 1 wherein the IgA antibody is a secretory IgA antibody.

3. The method of claim 1 wherein said administering is by sublingual injection, deposition, bioadhesive, or capsule that releases the immunogenic composition in the floor of the mouth.

4. The method of claim 3 wherein the bioadhesive or capsule contains or is coupled to a system that promotes penetration of the immunogenic composition.

5. The method according to claim 1 wherein the immunogen is from a pathogen chosen from the group consisting of HIV virus, herpesviruses, candidae, hepatitis viruses, picornaviridae, reoviruses, adenoviruses, human papillomavirus, cytomegalovirus, Epstein-Barr virus, and aerosol transmitted pathogens.

6. The method according to claim 5, wherein the aerosol transmitted pathogen is selected from the group consisting of Mycobacterium tuberculosis, Neisseria meningitidis, Streptococcus type B, Streptococcus pneumoniae and Bordetalla pertussis.

7. The method according to claim 6, wherein the herpesvirus is herpes simplex virus, the hepatits virus is hepatitis A, the picornaviridae are poliomyelitis virus, the reovirus is a rotavirus.

8. The method according to claim 1, wherein the immunogen is from the HIV virus.

9. The method according to claim 8, wherein the immunogen is gp160.
